(19) United States
(12) Patent Application Publication
(10) Pub. No.: US 2006/0030879 A1
(43) Pub. Date: Feb. 9, 2006

(54) System and method for applying acupressure via clothing

(76) Inventor: Donald Spector, New York, NY (US)

Correspondence Address: WILLIAM COLLARD;COLLARD & ROE, P.C., 1077 NORTHERN BOULEVARD, ROSLYN, NY, 11576, US

(21) Appl. No.: 10/913,206

(22) Filed: Aug. 6, 2004

(51) Int. Cl. A61B 17/00

(52) U.S. Cl. 606/204

(57) ABSTRACT

A method of applying acupressure to a person, comprises determining at least one specific area on an article of clothing that, when worn by said person, covers a specific known acupoint on the body, and securing pellet in place on said one specific area with an adhesive tape. The wearer can perform acupressure on him or herself by pressing on the pellet, which applies pressure to the specific acupoint.

### BACKGROUND OF THE INVENTION

1. Field of the Invention

This invention relates in general to a system and method or applying acupressure to a patient via articles of clothing having pellets adhered with adhesive tape. When the clothing is worn, the pellet is centered over and in close proximity to known pressure points including those points wherein acupuncture is customarily applied. Discretely applying pressure to the pressure point by means of the pellet, provides certain therapeutic benefits.

2. The Prior Art

Acupuncture is a well known practice of Chinese origin of puncturing the body with needles at specific points to relieve pain and effect the curing of certain diseases. The specific points, or acupoints, are well documented in the literature and known to practitioners in this art. Shiatsu is massage therapy applied with the fingers to those specific areas of the body used in acupuncture. Hence, when pressure is applied to shiatsu meridian lines on the human body, certain desirable benefits are obtained. The meridian are known as the pathway of life energy of the human body.

The present invention utilizes acupoints of oriental medical theory, the same acupoints to which acupuncture is applied. It is well known that there are acupoints located at specific positions of the human body and stimulations of a specific acupoint can result in medical benefits such as relieving pain, improved metabolism and improved health.

Over the past many centuries, the benefits of acupressure and massage have been well documented. Massage as a form of therapy can be traced back to the ancient Greeks and Romans as an accepted form of medical treatment. One form of massage, acupressure, has also been employed by the Chinese for over 5,000 years.

While the end result of acupressure and acupuncture are quite similar, they are different in that acupressure in its truest sense does not utilize needles. In either case it is believed that both systems stimulate the nervous system which releases neurohormones and affects the amounts of neurotransmitters. Moreover, both systems are effective for reducing muscle tension and pain, for at least some period of time. In addition to acupressure and acupuncture, the use of magnetism is also currently in vogue for the treatment of a variety of physical and other conditions.

More recently several methods and various kinds of apparatus have been proposed for applying pressure to the human body to provide relief from pain, stress and/or muscle tension. For example, U.S. Pat. No. 6,241,693 which issued Jun. 5, 2001 to Brian D. Lambden discloses a method and apparatus for applying pressure to the body of a person, particularly, those areas of the body which may be difficult to reach. The apparatus disclosed in this invention is comprised of an arcuate shaft, a handle that extends from the arcuate shaft in a plane intersecting the plane of the arcuate shaft, and a pressure applicator movably attached to the arcuate shaft for applying pressure to the body. The apparatus is used for massaging or for applying acupressure.

A magnetic acupuncture device which is adapted as a substitute for the conventional acupuncture needle is disclosed in U.S. Pat. No. 6,113,530 which issued to Nai-Hsin Chien on Sep. 5, 2000. The device is in the form of a magnetic acupuncture pen which has a head providing an acupuncture point adapted to massage and press on a specific acupoint on a human body to provide stimulations.

U.S. Pat. No. 6,299,586 which issued Oct. 9, 2001, to Thauh D. Cao, discloses an acupressure point treatment apparatus which includes a pellet on a finger strap. The strap includes a band extending around a finger and a rigid extension containing a pellet perpendicular to the axis of the fingertip of an acupressurist for performing acupressure treatment. The patent also discloses a galvanic skin response monitor electronically connected to the pellet. In one embodiment, the pellet can be made of silver or a magnetized material to provide an effect of magnetic therapy.

Therapeutic clothing has been described in U.S. Pat. No. 5,950,239, which issued Sep. 14, 1999 to Richard Lopez and contains a permanent magnet whereby the polarity is directed to selected locations of the body to assist in healing. The magnets are thin members and are positioned to contact the skin of the user. The magnets can be contained in various articles of clothing such as shirts, gloves, trousers, socks, brassieres, knee pads and the like.

In U.S. Pat. No. 6,261,306, which issued Jul. 17, 2001 to George Henry Kramer, an acupressure treatment device is disclosed for use on a selected skin surface portion of a human body. The device which can be in the form of a patch is made of a thin flexible sheet having a first side, a second side and a margin, wherein the margin is shaped to correspond to a selected skin surface portion for properly orienting the device. The first side contains an adhesive layer for attachment to the skin. At least one bead is located on the adhesive layer suitable for positioning the bead against the skin to provide acupressure to at least a portion of the body surfaces. In this device the patch with bead adheres to the skin until removal. Additionally, each time the path is replaced, it must be positioned for proper orientation over the acupoint.

However, prior to the present invention the application of acupressure, and particularly acupuncture, was usually done by a professional person at specific locations, such as a doctor's office, health club, or similar location. A person seeking benefit of such therapy such as acupuncture usually found difficulties in attempting to treat him or herself.

### SUMMARY OF THE INVENTION

Accordingly, one or more of the following objects will be achieved by the practice of this invention. It is an object of this invention to provide a method whereby a person seeking the benefit of acupressure, can him or herself apply such pressure to known pressure points on his or her own body with essentially complete discretion and for the most part, without another person being aware of such action. It is a further object of the invention to provide articles of clothing whereby a person can discretely apply pressure through such clothing to predetermined acupoints on one's body. Another object is to provide clothing which is custom tailored to have one or more regions or pouches incorporated into such clothing and when the clothing is worn, the region or pouch is located over one or more acupressure points on the wearer's body. A still further object is to provide customized clothing where the pouches contain pellets through which pressure can be applied by the wearer to certain pressure points resulting in a therapeutic benefit. Another object of the present invention is to provide clothing with pouches or regions which contain pellets which can be magnetized. Another object is to provide an adhesive tape, acupressure pellets and instructions regarding acupoints, whereby the individual can apply the acupressure pellets to their own clothing. There are other objects that will become readily apparent to the one skilled in the art in light of the teachings herein set forth.

In its broad aspects, the present invention is directed to method for applying acupressure to an acupoint on a human being and to articles of clothing having (a) at least one region of a size and shape which, when worn by a person, will center the region over and in-close proximity to a specific known pressure point on the body of the person wearing said clothing, and (b) located at such region at least one pellet of spherical or elliptical configurations, whereupon pressuring the pellet against the body of the person wearing the clothing, experts pin-point pressuring at the site of a known pressure point, providing at least some therapeutic benefit to said person. If desired, the pellet can be magnetized to obtain additional benefits. In another embodiment, the invention comprises an adhesive tape, an assortment of acupressure pellets, and instructions whereby the user, following the instructions, can apply the acupressure pellets via the tape to their own clothing in the region corresponding to certain acupressure points described in the instructions.

### BRIEF DESCRIPTION OF THE DRAWINGS

Other objects and features of the present invention will become apparent from the following detailed description considered in connection with the accompanying drawings. It is to be understood, however, that the drawings are designed as an illustration only and not as a definition of the limits of the invention.

In the drawings, wherein similar reference characters denote similar elements throughout the several views:

FIG. 1 and FIG. 2 depict the front and back view of a human body showing meridian lines and a few acupoints;

FIG. 3 is a view of a shirt;

FIG. 4 is a view of a pair of trousers;

FIG. 5 is a view of a person wearing a baseball cap and who is applying pressure at an acupoint on his forehead by pressing on pellet embedded in the cap;

FIG. 6 is a cross-sectional view of a garment showing a pouch and a pellet contained therein;

FIG. 7 is a view of the acupressure pellet and adhesive tape according to the invention; and

FIG. 8 is a view of an article of clothing having the acupressure pellets taped thereto.

### DETAILED DESCRIPTION OF THE PREFERRED EMBODIMENT

Although many of the known methods used for acupressure apply pressure over a wide area of the body without limiting the pressure to specific pressure points, the present invention is useful for pin-pointing applications of pressure just to those pressure points which are known to cause specific forms of relief. Accordingly, the present invention in its application is more like acupuncture than a massage since pressure is concentrated at specific points. Moreover, the pressure is concentrated to an even smaller area since it is transmitted through a spherical or elliptical pellet where the surface area to which pressure is applied is very small.

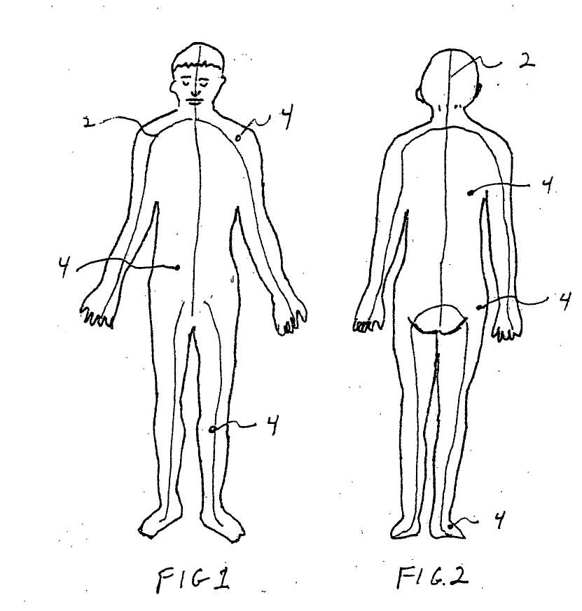

With respect to the drawings, FIG. 1 and FIG. 2 are front and back views of a human body showing meridian lines 2 and some shiatsu body points 4. It is understood that the specific location of acupoints on a person will, of course, vary from person to person, and hence the drawings are illustrative and only depict a few acupoints.

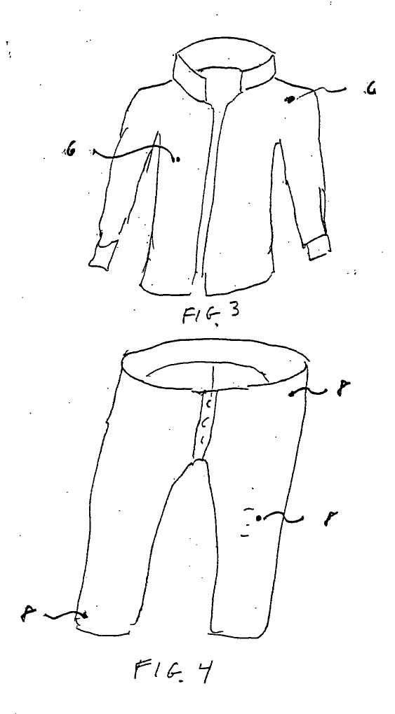

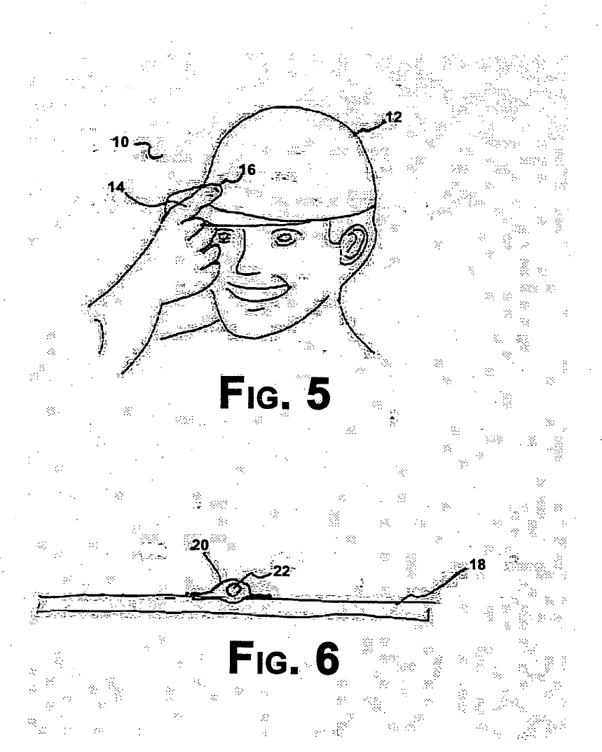

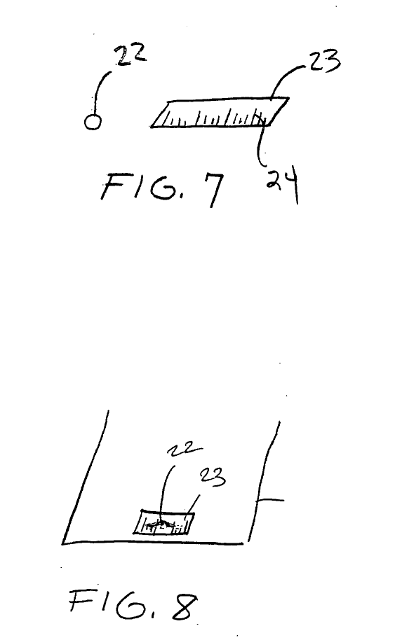

FIG. 3 and FIG. 4 merely show a shirt and trousers and point out acupoint 6 and 8 as typical locations on the garments wherein the pellets might be positioned.

FIG. 5 depicts an individual 10 wearing a cap 12 and pressing with a finger 14 on the exterior of the cap at acupoint 16 so that pressure is exerted through a pellet, (not shown), embedded in the cap material to the underlying acupoint.

FIG. 6 shows a cross-sectional view of a portion of a garment 18 showing a pouch 20 sewn into the garment and containing pellet 22.

Pellet 22, as shown in FIGS. 7 and 8, can be directly attached to an article of clothing via an adhesive tape 23, thus allowing the user to turn any article of clothing into acupressure clothing, such as shown in FIG. 8. Tape 23 is preferably relatively thick, i.e., preferably at least 2 mm, so as to cushion the user's body against rubbing caused by the protruding pellet. The object is to create sufficient pressure so as to be able to perform acupressure, but not have so large a protrusion so as to be noticeable or uncomfortable. Tape 23 is preferably marked with ruler markings (24), so as to assist the user in measuring the amount of tape needed as well as the placement of the tape and the pellet.

The acupoints on the body at which acupressure is applied are well known points throughout the human body and are those where acupuncture needles are usually inserted. For example, U.S. Pat. No. 5,950,239 discloses in FIG. 1 and FIG. 2 the frontal and backside views of a human body showing meridian lines and shiatsu body points.

U.S. Pat. No. 6,299,586 indicates in a discussion of background art that acupuncture and acupressure points can be located with a point locator which includes a low-current galvanometer, which is commonly known to an acupuncturist as a Galvanic Skin Response Monitor or to bio-feedback therapists, as a Skin Conductor Monitor. The treatment point is located in the region where electrical resistance of the skin is relatively lower than that of the surrounding tissues. Accordingly, for any one individual the exact location of acupoints on such person's body can easily be determined. The directions accompanying the tape and pellets provide diagrams and measurements as to the location of the specific acupoints on the body, so as to allow the user to position the pellets at the most appropriate places on their own clothing.

As noted above, the present invention is directed to an acupressure system and method using articles of clothing to which pellets are positioned via adhesive tape or other methods, so that when the clothing is worn, the pellets are located over acupoints and pressure can be applied by the wearer to the pellet and at the acupoint.

The types of clothing which can be employed in the present invention cover a wide variety of styles, fabrics and designs. The main characteristic is that the clothing be tailored such that when it is worn, the pouch or the area in which the pellet is embedded is in alignment with the pressure point and when pressure is applied to the pellet, it is at the proper locations to be in alignment with the known pressure point.

Hence, the term “clothing” as used herein is intended to cover most any article of clothing which a person can wear and which covers one or more acupressure points on the human body. Illustrative articles of clothing include but are not limited to, dress shirts, gloves, trousers, socks, suits, brassieres, caps, skirts, T-shirts, briefs, boxer shorts, belts, blouses, sport shirts, bathing suits, jackets, coats, and the like.

The term “customized clothing” as used throughout the specification and claims is intended to cover clothing which has one or more regions or pouches included in the clothing when it is made. It also is intended to include clothing which has pouches or other means added to, or affixed on, the clothing after it has been fabricated to contain the pellets.

The only limitation on the type of clothing that can be customized to have the pellet-containing pouch is the location of the pressure points on the human body. For example, if the pressure points are on the head, appropriate clothing can include caps, hats, hoods, sweat bands, and the like. If the pressure point is on the neck or shoulder, the pellets can be located in the collar or back, side or front of the shoulder area of a sport or dress shirt or a turtle-neck shirt.

The pouch itself can be in the form of a pocket, bag, sack etc., or even a small pocket within a large pocket for retention of the pellet. Hence, the pouch should be small enough so that the pellet is confined to a specific area of the clothing, yet of such configuration so that the pellet can be removed if desired when the clothing is washed or dry-cleaned. Openings in the pouch can be sealable after removal and/or replacement of the pellet by VELCRO® hook-and-loop type fasteners or other means. If the clothing is of such form, or if the pellet is small enough so that removal is not necessary, the pellet can be permanently embedded in the clothing fabric.

At the time the clothing is made, such as a custom made suit, the fabric can be sewn to incorporate the pellets at specific locations within the fabric layers. Although the pellets could be permanently sewn into the clothing, for practical purposes, such as pressing, it is desirable to have means for removing and replacing the pellets. For example, Velcro fasteners could be used to seal and unseal a small pouch possibly in the lining of the clothing. In the event that it is desired to attach a pellet to ready made clothing, this can be done by also attaching a small pouch with the pellet by several means such as Velcro strips.

The term “pellet” as used throughout the specification and appended claims is intended to encompass metal or non-metal (plastic, marble, etc.) items which are substantially in the shape of a ball, sphere, spherical, bead, shot, marble, pea, pebble, seed and the like. Excluded are objects which have large flat surfaces. The shape of the pellet should be such that when pressed against the body, pressure is exerted in a very localized area, i.e., a pin-point, as would be done by an acupuncture needle. Hence, the curvature of the pellet is important so that a minimum of surface area touches the body for a maximum of applied pressure.

The pellets themselves can be made of a variety of metals, plastics, ceramics or even in some cases wood. For practice, however, it is preferred that the pellet be metal, preferably spherical in shape, and made of steel, iron, a metal alloy, nickel, silver, and the like. The pellet can also be glass, ceramic, marble, plastic and the like. If made of wood, it should be a hard wood such as maple, and preferably spherical in shape.

The size of the pellet will largely be determined by the clothing in which it is embedded as well as the location of the acupressure point. Pellets of three-eighths to one quarter inch or less in diameter would not be conspicuous or show through the clothing and yet be large enough when pressed to apply the necessary pressure to the acupoint. Of course, larger pellets or pellets of elliptical shape can be used if desired even those of one-half inch in diameter or larger.

The present invention differs from known wrist bands which had been available in the past, since the pressure inducing means on such bands were not embedded or concealed in a band and usually were button shaped whereby pressure was not concentrated or pin-pointed to a very specific area. Moreover, as long as the wrist band was worn, pressure was being applied.

An advantage of the present invention is that the person wearing the therapeutic clothing can apply pressure to one or more acupoints on his or her body even in public without anyone else being aware of what is happening. It can be done very discretely and no one would know that a person is receiving acupressure treatment by simply pressing on a certain area of a cap, shirt, trouser or other garment. The person need only feel through the clothing until the pellet is touched and then press the pellet to the skin for whatever period is necessary to achieve at least some of the desired results.

In a further embodiment of the present invention, it has been found that additional therapeutic benefits can be obtained if the pellet which is embedded in the clothing has been magnetized so as to provide additional benefit to the person wearing the clothes.

In yet another embodiment of the invention, the pellet can be applied to an existing piece of clothing via an adhesive tape. Thus, the clothing does not have to be altered or specially sewn. In this embodiment, the user is provided with instructions regarding the proper placement of the pellet on the clothing, and special tape that will adhere to the clothing and hold the pellet in place. The proper placement of the pellet is aided by tape measure or ruler markings on the non-adhesive side of the tape. These markings can be used to measure out the distance from the edge or center of the article of clothing to the area corresponding to the desired acupoint, to indicate proper placement of the pellet.

Although the invention has been illustrated by the preceding description, it is not to be construed as being limited to the materials disclosed herein. The invention is directed to the generic areas as herein before disclosed. Various modifications and embodiments thereof can be made without departing from the spirit and scope thereof.

Accordingly, while only a few embodiments of the present invention have been shown and described, it is obvious that many changes and modifications may be made thereunto without departing from the spirit and scope of the invention.

What is claimed is:

1. A method of applying acupressure to a person, comprising:
determining at least one specific area on an article of clothing that, when worn by said person, covers a specific known acupoint on the body;
placing a pellet on a length of adhesive tape; and
securing the pellet in place over said at least one specific area with the adhesive tape.

2. The method according to claim 1, wherein the pellet is spherical or elliptical.

3. The method according to claim 1, wherein the tape is smooth on its non-adhesive side.

4. The method according to claim 1, further comprising the steps of placing the article of clothing on the person's body, and pressing the pellet on the acupoint for a predetermined amount of time.

5. The method according to claim 1, wherein the adhesive tape has a thickness of at least 2 mm.

6. The method according to claim 1, wherein the adhesive tape has an adhesive side and a smooth side, and wherein the smooth side is imprinted with ruler markings.

7. A system for allowing an individual to perform acupressure on themselves, comprising:
at least one acupressure pellet;
adhesive tape sufficient to adhere the pellet to an article of clothing; and
directions indicating where on a person's body various acupressure points are located, so that the person can adhere the pellet to their clothing in an area where the pellet is located directly adjacent one of the acupressure points when the article of clothing is being worn, so that the person can perform acupressure by pressing the pellet against their body.

8. The system according to claim 7, wherein the pellet is round or elliptical and has a diameter of between about ½ inch and 1 inch.

9. The system according to claim 7, wherein the adhesive tape has a width of about between 1 inch and two inches.

10. The system according to claim 7, wherein the directions are printed directions.

11. The system according to claim 7, wherein the directions are in the form of a video on a DVD, CD-ROM or videocassette.

12. The system according to claim 7, wherein the tape is printed with a tape measure on its non-adhesive side.

13. The system according to claim 7, wherein the tape has a thickness of at least 2 mm.
